SEQUENCE LISTING (8 sequences)

SEQ ID NO: 1 - protein, 108 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 10 aa, Homo sapiens
GFTFSRYWMS

SEQ ID NO: 4 - protein, 17 aa, Homo sapiens
NIKQDGSEKYYVDSVKG

SEQ ID NO: 5 - protein, 12 aa, Homo sapiens
EGGWFGELAFDY

SEQ ID NO: 6 - protein, 12 aa, Homo sapiens
RASQRVSSSYLA

SEQ ID NO: 7 - protein, 7 aa, Homo sapiens
DASSRAT

SEQ ID NO: 8 - protein, 9 aa, Homo sapiens
QQYGSLPWT

(19) United States
(12) Patent Application Publication
(10) Pub. No.: US 2021/0054079 A1
(43) Pub. Date: Feb. 25, 2021

(54) FORMULATIONS OF HUMAN ANTI-PD-LI ANTIBODIES

(71) Applicant: MEDIMMUNE LIMITED, Cambridge, Cambridgeshire (GB)

(72) Inventors: James Biddlecombe, Cambridge, Cambridgeshire (GB); Jenny Main, Gaithersburg, MD (US); Jiali Du, Gaithersburg, MD (US); Methal Albarghouthi, Gaithersburg, MD (US)

(21) Appl. No.: 17/049,839

(22) PCT Filed: Apr. 24, 2019
(86) PCT No.: PCT/EP2019/060498

§ 371 Date: Oct. 22, 2020

Related U.S. Application Data
(60) Provisional application No. 62/662,324, filed on Apr. 25, 2018.

(51) Int. Cl. C07K 16/28; A61K 47/26

(52) U.S. Cl. CPC C07K 16/2827; A61K 47/26; C07K 2317/41; C07K 2317/565; C07K 2317/94

(57) ABSTRACT

This disclosure relates to formulations and compositions of an antibody directed against human anti-PD-L1, such as durvalumab.

### BACKGROUND

### 1. Field of the Invention

This disclosure relates to formulations and compositions of an antibody directed against human anti-PD-L1, such as durvalumab.

### 2. Background

Programmed death-ligand 1(PD-L1), also known as B7H1, is a 40 kDa transmembrane protein that provides a major obstacle to anti-cancer immunity. PD-L1 binding to the programmed death receptor (PD-1) inactivates T-cells, protects tumor cells, and suppresses immune system detection, allowing for unchecked proliferation of cancer cells. PD-L1 also binds CD80, a co-stimulatory molecule.

A wide range of tumorigenic and activated immune cell types naturally express PD-L1, including antigen presenting cells, macrophages, monocytes, B cells, T cells, and non-hematopoietic cells. In addition, inflammatory cytokines, such as interferon gamma (IFNγ), can induce PD-L1 expression. For example, activated T-cells produce IFNγ, which is the most potent inducer of PD-L1. PD-L1 expression induced by IFNγ promotes tumor protection, which is a mechanism known as adaptive immune resistance.

One strategy for combating adaptive immune resistance, and the lethality of PD-L1, is with anti-PD-L1 antibodies. Consistent with this approach, the anti-PD-L1 antibody durvalumab, which is a 149 kDa, affinity optimized anti-PD-L1 monoclonal IgG1 triple mutant (TM) that disrupts PD-L1 binding to PD-1, can be employed to eliminate the immunosuppressive effects of PD-L1 on cytotoxic T cells. The result is mitigation of the negative inhibitory signals that promote tumor growth and enhance anti-tumor immunity, responses that enhance tumor cell killing by the immune system.

Importantly, anti-PD-L1 antibody buffer formulations that retain liquid drug substance and lyophilized drug product stability are essential to the effectiveness of anti-PD-L1 antibodies.

### SUMMARY

In one aspect, the disclosure provides an antibody formulation comprising 40 mg/mL to 50 mg/mL of an anti-PD-L1 antibody, 15 mM to 35 mM buffer, 255 mM to 275 mM disaccharide, 0.01% (w/v) to 0.05% (w/v) surfactant and wherein the pH of the formulation is about pH 5.5 to about pH 7.2.

In one aspect, the disclosure provides an antibody formulation comprising 50 mg/mL of a human anti-PD-L1 antibody, 26 mM histidine/histidine-HCl buffer, 275 mM trehalose dehydrate, 0.02% (w/v) polysorbate 80 and wherein the pH of the formulation is pH 6.0.

In one aspect, the disclosure provides an antibody formulation comprising 50 mg/mL of a human anti-PD-L1 antibody, 25 mM histidine/histidine-HCl buffer, 265 mM trehalose dehydrate, 0.02% (w/v) polysorbate 80 and wherein the pH of the formulation is about pH 5.5.

In one aspect, the disclosure provides an antibody formulation comprising 50 mg/mL of a human anti-PD-L1 antibody, 25 mM a histidine/histidine-HCl buffer, 265 mM trehalose dehydrate, 0.02% (w/v) polysorbate 80 and wherein the pH of the formulation is about pH 6.5.

In one aspect, the disclosure provides an antibody formulation comprising 50 mg/mL of a human anti-PD-L1 antibody, 25 mM a histidine/histidine-HCl buffer, 265 mM trehalose dehydrate, 0.04% (w/v) polysorbate 80 and wherein the pH of the formulation is about pH 6.0.

In one aspect, the disclosure provides an antibody formulation comprising 50 mg/mL of a human anti-PD-L1 antibody, 25 mM a histidine/histidine-HCl buffer, 265 mM sucrose, 0.02% (w/v) polysorbate 80 and wherein the pH of the formulation is about pH 6.0.

In one aspect, the disclosure provides a composition comprising an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2 and a main form of the antibody comprising greater than, or equal to, 45% of the protein in the composition as measured using capillary isoelectric focusing (cIEF) of the composition.

In one aspect, the disclosure provides a composition comprising an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2, a main form of the antibody comprising greater than, or equal to, 45% of the protein in the composition as measured using cIEF of the composition, and acidic forms of the antibody comprising 45% to 50% of the protein in the composition as measured using cIEF of the composition.

In one aspect, the disclosure provides a composition comprising an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2, a main form of the antibody comprising greater than, or equal to, 45% of the protein in the composition as measured using cIEF of the composition, and a basic form of the antibody comprising 18% to 23% of the protein in the composition as measured using cIEF of the composition.

In one aspect, the disclosure provides a composition comprising an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2, a main form of the antibody comprising greater than, or equal to, 45% of the protein in the composition as measured using cIEF of the composition, acidic forms of the antibody comprising 45% to 50% of the protein in the composition as measured using cIEF of the composition, and a basic form of the antibody comprising 18% to 23% of the protein in the composition as measured using cIEF of the composition.

In one aspect, the disclosure provides a composition comprising an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2 and the glycan structures of the anti-PD-L1 antibody comprise G0f, G1f, G2f, and G0 glycoforms.

In one aspect, the disclosure provides a composition comprising an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2, 1.5%-2.5% of the anti-PD-L1 antibody forms an aggregate as determined by high-pressure size exclusion chromatography (HP-SPEC), and 97%-98% of the anti-PD-L1 antibody is present as a monomer as measured by HP-SEC.

### BRIEF DESCRIPTION OF THE DRAWINGS

FIG. 1 shows a schematic of the durvalumab formulation development activities.

FIG. 2 shows a Differential Scanning calorimetry (DSC) profile of durvalumab at 3 mg/mL in the formulation buffer (26 mM histidine/histidine-HCl, 275 mM trehalose dehydrate, 0.02% (w/v) polysorbate 80, pH 6.0), wherein Tm₁ was demonstrated to be 64.5° C. and Tm₂ was demonstrated to be 73.04° C.

FIG. 3 is a capillary Isoelectric focusing (cIEF) profile of durvalumab in the formulation buffer (26 mM histidine/histidine-HCl, 275 mM trehalose dehydrate, 0.02% (w/v) polysorbate 80, pH 6.0). The four peaks displayed a pI range between 8.3 and 8.8. The main peak had a pI of 8.6.

FIG. 4 demonstrates the liquid stability of durvalumab clone 1 or clone 2 after incubation for 1 month at 5° C. or 40° C. in the formulation buffer (26 mM histidine/histidine-HCl, 275 mM trehalose dehydrate, 0.02% (w/v) polysorbate 80, pH 6.0).

FIGS. 5A-5C depict electropherograms of durvalumab after three months of storage at 5° C. (FIG. 5A), 25° C. (FIG. 5B), and 40° C. (FIG. 5C). There was an additional pyroglutamic acid peak after 3 months of storage at 25° C. and 40° C.

FIG. 6 demonstrates freeze-dry microscopy showing onset of collapse and full collapse temperature of durvalumab in the formulation buffer (26 mM histidine/histidine-HCl, 275 mM trehalose dehydrate, 0.02% (w/v) polysorbate 80, pH 6.0).

FIG. 7 depicts a DSC thermogram showing the glass transition (Tg′) temperature of durvalumab in the formulation buffer (26 mM histidine/histidine-HCl, 275 mM trehalose dehydrate, 0.02% (w/v) polysorbate 80, pH 6.0).

FIG. 8 is a design space generated process model. The experimental design runs altered primary drying temperature, pressure, primary drying time, and max product temperature. The cycle midpoint (dot) and process robustness (light, triangle shaped area) around chamber pressure and shelf temperature are denoted.

FIG. 9 demonstrates durvalumab NMF lyophilization run data in which a convergence of pirani gauge (delta 10 μbar) occurs at approximately 103 hours into the 115-hour drying step. This is equivalent to a 10% safety margin.

FIG. 10 demonstrates a larger scale NMF lyophilization run in the Amsco freeze-dryer.

Results showed convergence of the product thermocouples within the allotted primary time.

FIG. 11 demonstrates micro-flow imaging (MFI) results of durvalumab shaking studies.

FIG. 12 demonstrates Ultra Performance Liquid Chromatography (UPLC) peak identification of 2-AB labeled oligosaccharides on durvalumab.

### DETAILED DESCRIPTION

This disclosure relates to formulations and compositions of an antibody directed against anti-PD-L1, such as durvalumab.

As utilized in accordance with the present disclosure, the following terms, unless otherwise indicated, shall be understood to have the following meanings. Unless otherwise required by context, singular terms shall include pluralities and plural terms shall include the singular.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain” as used herein refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a naturally occurring antibody, a full-length heavy chain immunoglobulin polypeptide includes a variable domain (VH) and three constant domains (CH1, CH2, and CH3) and a hinge region between CH1 and CH2, wherein the VH domain is at the amino-terminus of the polypeptide and the CH3 domain is at the carboxyl-terminus, and a full-length light chain immunoglobulin polypeptide includes a variable domain (VL) and a constant domain (CL), wherein the VL domain is at the amino-terminus of the polypeptide and the CL domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The antibody formulations disclosed herein comprise an anti-PD-L1 antibody. In particular embodiments, the formulation comprises 30 mg/mL, 35 mg/mL, 40 mg/mL, 45 mg/mL, 50 mg/mL, 55 mg/mL, or 60 mg/mL of the anti-PD-L1 antibody. In other embodiments, the formulation comprises 40 mg/mL to 50 mg/mL of the anti-PD-L1 antibody. In other embodiments, the formulation comprises 50 mg/mL of the anti-PD-L1 antibody. In particular embodiments, the anti-PD-L1 antibody is human.

The antibody formulations disclosed herein comprise one or more buffers. As used herein, “buffer” refers to an excipient for maintaining the pH of a formulation. In particular embodiments, the buffer is histidine/histidine-HCl buffer. The buffer is present at a concentration of about 15 mM, 20 mM, 25 mM, or 30 mM. In particular embodiments, the buffer concentration is 26 mM.

In particular embodiments, the antibody formulations comprise a disaccharide. In certain embodiments, the disaccharide is trehalose dihydrate or sucrose. The disaccharide is present at a concentration of about 250 mM, 255 mM, 260 mM, 265 mM, 270 mM, 275 mM, or 280 mM. In particular embodiments, the disaccharide concentration is 265 mM. In other embodiments, the disaccharide concentration is 275 mM.

In particular embodiments, the antibody formulations comprise a surfactant. The term “surfactant” as used herein refers to organic substances having amphipathic structures; namely, such substances are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical formulations and preparations of biological materials. In particular embodiments, the surfactant is polysorbate 80. The surfactant is present at a concentration of about 0.001% to about 0.5% (by volume).

In particular embodiments, disclosed herein is an antibody formulation comprising 40 mg/mL to 50 mg/mL of an anti-PD-L1 antibody, 15 mM to 35 mM buffer, 255 mM to 275 mM disaccharide, 0.01% (w/v) to 0.05% (w/v) surfactant, and wherein the pH of the formulation is about pH 5.5 to about pH 7.2. In particular embodiments the antibody formulation comprises 50 mg/mL of a human anti-PD-L1 antibody, 26 mM histidine/histidine-HCl buffer, 275 mM trehalose dehydrate, 0.02% (w/v) polysorbate 80, and wherein the pH of the formulation is about 6.0. In other embodiments, the antibody formulation comprises 50 mg/mL of a human anti-PD-L1 antibody, 25 mM histidine/histidine-HCl buffer, 265 mM trehalose dehydrate, 0.02% (w/v) polysorbate 80, and wherein the pH of the formulation is about 5.5. In other embodiments, the antibody formulation comprises 50 mg/mL of a human anti-PD-L1 antibody, 25 mM histidine/histidine-HCl buffer, 265 mM trehalose dehydrate, 0.02% (w/v) polysorbate 80, and wherein the pH of the formulation is about 6.5. In other embodiments, the antibody formulation comprises 50 mg/mL of a human anti-PD-L1 antibody, 25 mM histidine/histidine-HCl buffer, 265 mM trehalose dehydrate, 0.04% (w/v) polysorbate 80, and wherein the pH of the formulation is about 6.0. In other embodiments, the antibody formulation comprises 50 mg/mL of a human anti-PD-L1 antibody, 25 mM histidine/histidine-HCl buffer, 265 mM sucrose, 0.02% (w/v) polysorbate 80, and wherein the pH of the formulation is about 6.0. In other embodiments, the antibody formulation comprises 50 mg/mL of a human anti-PD-L1 antibody, 25 mM histidine/histidine-HCl buffer, 265 mM sucrose, 0.02% (w/v) polysorbate 80, and wherein the pH of the formulation is about 6.0.

In particular embodiments, the human anti-PD-L1 antibody comprises a light chain variable domain comprising the amino acid sequence of SEQ ID NO: 1 and a heavy chain variable domain comprising the amino acid sequence of SEQ ID NO: 2. In other embodiments, the human anti-PD-L1 antibody comprises a VH CDR1 having the amino acid sequence of SEQ ID NO: 3, a VH CDR2 having the amino acid sequence of SEQ ID NO: 4, a VH CDR3 having the amino acid sequence of SEQ ID NO: 5, a VL CDR1 having the amino acid sequence of SEQ ID NO: 6, a VL CDR2 having the amino acid sequence of SEQ ID NO: 7, and a VL CDR3 having the amino acid sequence of SEQ ID NO: 8.

In particular embodiments, the human anti-PD-L1 antibody comprises a light chain variable domain comprising an amino acid sequence that is less than 100% identical to the amino acid sequence of SEQ ID NO: 1 and a heavy chain variable domain comprising an amino acid sequence that is less than 100% identical to the amino acid sequence of SEQ ID NO: 2. In other embodiments, the human anti-PD-L1 antibody comprises a light chain variable domain comprising an amino acid sequence having 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity to the amino acid sequence of SEQ ID NO: 1 and a heavy chain variable domain comprising an amino acid sequence having 90% sequence identity, 91% sequence identity, 92% sequence identity, 93% sequence identity, 94% sequence identity, 95% sequence identity, 96% sequence identity, 97% sequence identity, 98% sequence identity, or 99% sequence identity to the amino acid sequence of SEQ ID NO: 2.

The terms “MEDI4736” and “durvalumab” as used herein refer to an antibody that selectively binds human anti-PD-L1 and blocks the binding of PD-L1 to PD-1 and CD80 receptors, as disclosed in U.S. Pat. Nos. 8,779,108 and 9,493,565, which are each incorporated by reference herein in their entireties. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (ADCC). Durvalumab can relieve PD-L1-mediated suppression of human T-cell activation in vitro and inhibits tumor growth in a xenograft model via a T-cell dependent mechanism.

As used herein, the phrases “pharmaceutical formulation,” “formulation,” and “antibody formulation” are used interchangeably and refer to a composition comprising an anti-PD-L1 antibody and one or more appropriate buffers and/or excipients. Suitably, the pharmaceutical formulations described herein are “pharmaceutically acceptable,” and thus would meet the necessary approval requirements required by a regulatory agency of the Federal or a state government, or listed in the U.S. Pharmacopeia, European Pharmacopeia, or other generally recognized pharmacopeia, so as to be used in animals, and more particularly in humans.

The antibody formulations disclosed herein can be formulated as a liquid formulation, a frozen formulation, a lyophilized formulation, or a reconstituted formulation.

Lyophilization can occur via drying in an oven, vacuum centrifugation, or other means known by one skilled in the art. Lyophilized durvalumab retains activity of the anti-PD-L1 antibody when reconstituted.

The terms “stability” and “stable” as used herein in the context of a formulation comprising an anti-PD-L1 antibody refer to the resistance of the antibody in the formulation to aggregation, degradation, or fragmentation under given manufacture, preparation, transportation, and storage conditions. The “stable” formulations retain biological activity under given manufacture, preparation, transportation, and storage conditions. The stability of the antibody can be assessed by degrees of aggregation, degradation, or fragmentation, as measured by high-pressure size exclusion chromatography (HP-SEC), static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. The overall stability of a formulation comprising a human PD-L1 antibody can be assessed by various immunological assays including, for example, ELISA and radioimmunoassay using isolated antigen molecules.

In particular embodiments, less than about 1% of the anti-PD-L1 antibody forms an aggregate upon storage at 40° Celsius for about 1 month as determined by HP-SEC. In other embodiments, at least 97% of the human anti-PD-L1 antibody is present as a monomer following storage at about 40° Celsius for about 1 month as measured by HP-SPEC. In other embodiments, at least 99% of the human anti-PD-L1 antibody is present as a monomer following storage at about 40° Celsius for about 1 month as measured by HP-SPEC. In other embodiments, at least 98% of the human anti-PD-L1 antibody is present as a monomer following storage at about 5° Celsius for about 1 month as measured by HP-SPEC. In particular embodiments, an antibody formulation maintains stability following at least three freeze/thaw cycles.

In particular embodiments, a composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; and wherein a main form of the antibody comprises greater than, or equal to, 45% of the protein in the composition as measured using capillary isoelectric focusing (cIEF) of the composition. In other embodiments, a composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; wherein a main form of the antibody comprises greater than, or equal to, 45% of the protein in the composition as measured using cIEF of the composition; and wherein acidic forms of the antibody comprise 45% to 50% of the protein in the composition as measured using cIEF of the composition. In other embodiments, a composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; wherein a main form of the antibody comprises greater than, or equal to, 45% of the protein in the composition as measured using cIEF of the composition; and wherein a basic form of the antibody comprises 18% to 23% of the protein in the composition as measured using cIEF of the composition. In other embodiments, a composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; wherein a main form of the antibody comprises greater than, or equal to, 45% of the protein in the composition as measured using cIEF of the composition; wherein acidic forms of the antibody comprise 45% to 50% of the protein in the composition as measured using cIEF of the composition; and wherein a basic form of the antibody comprises 18% to 23% of the protein in the composition as measured using cIEF of the composition.

In particular embodiments, a composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; and wherein the glycan structures of the anti-PD-L1 antibody comprise G0f, G1f, G2f, and G0 glycoforms. In other embodiments, the glycan structures of the anti-PD-L1 antibody have a content greater than about 90% for the G0f, G1f, G2f, and G0 forms. In some embodiments, the composition of the anti-PD-L1 antibody comprises about 65-75% G0f content, 13-23% G1f content, 0-3% content G2f, and 0-4% G0 content. In other embodiments, the composition of the anti-PD-L1 antibody comprises about 71.9% G0f content, 18.4% G1f content, 1.5% content G2f, and 1.9% G0 content.

In particular embodiments, a composition disclosed herein comprises an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; wherein 1.5%-2.5% of the anti-PD-L1 antibody forms an aggregate as determined by high-pressure size exclusion chromatography (HP-SPEC); and wherein 97%-98% of the anti-PD-L1 antibody is present as a monomer as measured by HP-SEC. Without limiting the disclosure, a number of embodiments of the disclosure are described below for purpose of illustration. The Examples that follow are illustrative of specific embodiments of the invention, and various uses thereof. They are set forth for explanatory purposes only, and are not to be taken as limiting the invention.

### Example 1

### Materials and Methods

### 1. Protein Concentration Determination

Durvalumab protein concentrations were determined by measuring absorbance at 280 nm on an Aligent UV-V spectrophotometer. Dilutions were made in the formulation buffer. Protein concentrations were calculated by using a theoretical extinction coefficient of 1.55 (mg/mL)-1 cm-1. In a subset of determinations, the experimental coefficient of 1.52 (mg/mL)-1 cm-1 was used in the calculation.

### 2. High Pressure Size Exclusion Chromatography (HP-SEC) Analysis

Samples for HP-SEC analysis were first eluted isocratically with 0.1 M disodium phosphate containing 0.1 M sodium sulfate, pH 6.8, at a flow rate of 1.0 mL/minute. Eluted protein was detected at an absorbance of 280 nm. Results were reported as a percent area of the product monomer peak compared to all other peaks. The buffer-related peak observed at approximately 12 minutes was excluded from the reported results. Any peaks that eluted earlier than the monomer peak were recorded as percent aggregate. The peaks eluted after the monomer peak were recorded as percent fragments.

### 3. Visual Analysis

Visual inspection of the sample was performed by examining the glass vials for color, clarity, and the presence of particulate and fibrous matter using a light box with both light and dark backgrounds. Visible particles and clarity were assessed.

### 4. Visible Particle Analysis via High Accuracy (HIAC) Liquid Particle Counter

Samples were diluted to 5 mg/mL with filtered formulation buffer and then allowed to settle for 30 minutes prior to analysis. Samples were prepared in thrice-ultrapure water rinsed falcon tubes. Samples with a protein concentration <5 mg/mL were analyzed neat. Six readings for each sample were obtained using a Pacific Standard HIAC Royco 3000A and 8-channel particle counter 8000A. The average of the final three readings was recorded as the final sub-visible particle count. Cumulative particle counts >10-25 μm were recorded.

### 5. Osmolality and pH

Osmolality was measured using a Gonotec Osmomat 030-D Osmometer. The pH of the solution was measured using a PHM220 Lab pH meter.

### 6. Karl Fischer Analysis

The presence of residual water in the freeze-dried formulation was measured using Karl-Fischer titration (Mettler Toledo). Freeze-dried material was reconstituted using dry methanol. Residual water was determined based on the amount of water in the dry methanol and weight of the total solids.

7. Capillary Isoelectric Focusing (cIEF)

Samples were adjusted to 0.25 mg/mL with HPLC grade water. Samples were digested with Carboxypeptidase B (CBP) for 10 minutes at 37° C. then diluted with 1% methylcellulose solution, Pharmalyte pH 3-10, pI marker 9.46, and pI Marker 5.85. Samples were loaded onto an iCE280 Analyzer and focused at 1500 V for 1 minute, followed by 3000V for 7 minutes. The resulting electropherograms were analyzed using EZChrom software and compared to a reference standard.

### 8. Reducing and Non-Reducing Gel Electrophoresis

The Agilent 2100 BioAnalyzer with Protein 230 LabChip technology (Agilent) was used to analyze durvalumab by reducing and non-reducing gel electrophoresis. The LabChip channels allowed for separation, staining, de-staining, and detection. Samples and standard were adjusted to 4 mg/mL in PBS and mixed 1:1 with SDS denaturing sample buffer in the presence of 60 mM N-ethylmaleimide (non-reduced) or 60 mM dithiothreitol (reduced). Samples were then heated, centrifuged, diluted in water and loaded into a well on the LabChip. In the first dimension, proteins were separated with resolution comparable to a 4-20% gradient gel. Proteins were separated in the second dimension by molecular weight. A fluorescent dye, present in the sample buffer, was excited at 633 nm.

9. Differential Scanning calorimetry

Differential Scanning calorimetry (DSC) was used to determine melting temperature (Tm1).

### 10. Viscosity

Viscosity of samples and buffers was measured using an Anton Paar AMVn Viscometer. Measurements were made at target concentrations.

### 11. Amino-Acid Sequence Analysis for Hot Spots

The amino acid sequence of durvalumab was analyzed using BLAZE software to identify amino acid residues for hot spots or potential modification sites. Hot-spot reactivity was assigned a risk score of high, medium, or low congruent with sequence liability criteria, based on experience with other monoclonal antibodies.

### 12. Identification of N-Linked Oligosaccharides in Durvalumab

The N-linked oligosaccharides in durvalumab detected as significant peaks in the Ultra Performance Liquid Chromatography (UPLC) were identified using a Waters UPLC system with FLR detector. Durvalumab Reference Standard (10.2 mg/mL; 100 μg), formulated in 26 mM histidine/histidine-HCl, 275 mM trehalose dihydrate, 0.02% (w/v) polysorbate 80, pH 6.0, was reconstituted to 0.5 mg/mL in 50 mM Tris buffer (pH 7.8). Samples were digested with 2 μL PNGase F (Promega), labeled with fluorescent tag 2-aminobenzamide (2-AB; Sigma-Aldrich), cleaned with HILIC SPE cartridge, and eluted into water for UPLC profiling. The 2-AB labeled oligosaccharides were digested further with various exo-glycosides, including fucosidase, sialidase A, mannosidase, β-galactosidase, and β-N-Acetylhexosaminidase, for peak identification.

Samples were injected into an Acquity UPLC® BEH Glycan column (1.7 μM, 2.1×150 mm) on a Waters UPLC system using 50 mM ammonium formate, pH 4.4 as the mobile phase A, and 100% acetonitrile as the mobile phase B. Data was acquired and profiles of glycosidase digested samples were compared closely against non-digested samples to identify glycans and their oligosaccharide linkages.

### Example 2

### Developability Study

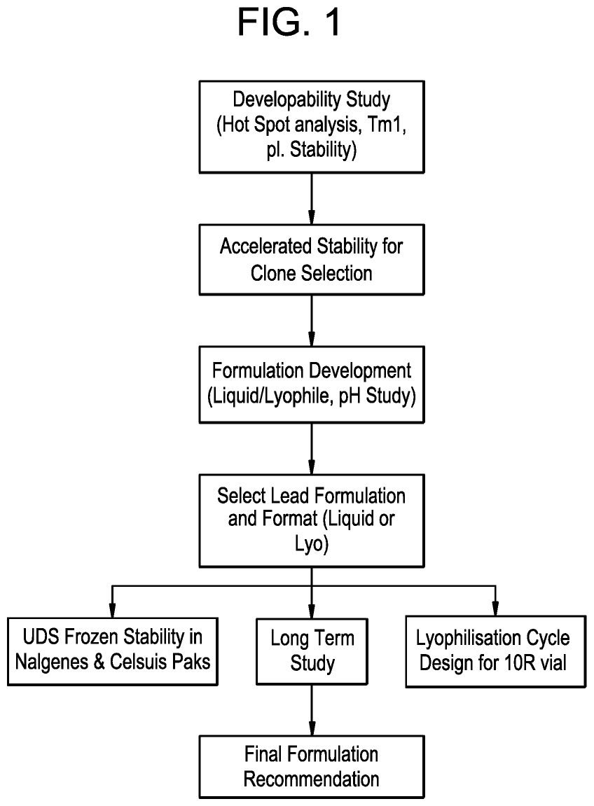

The durvalumab formulation was determined following a developability study, which included Hot Spot analysis, determination of melting temperature, isoelectric point (pI), and stability (FIG. 1).

### 1. Hot Spot Analysis

Durvalumab stability in storage is critical to effectiveness of the monoclonal antibody. A risk of antibody storage is the loss of activity, with the primary degradation route via aggregation. Additionally, amino acid residue modification from prolonged storage can influence stability and activity of the antibody.

Loss of activity in durvalumab was assessed by Homogenous Time Resolved Fluorescence (HTRF). No loss of activity was observed after incubation at 40° C. for 1 month in both high and low pH buffer. Only trace levels of Asp-isomerization at D54(G) were detected by peptide mapping after 1 month in a pH 6.0 buffer at 40° C.

2. Determination of Melting Temperature, Isoelectric Point (pI), and Stability

Melting point of the antibody was determined using Differential Scanning calorimetry (DSC) of durvalumab at 3 mg/mL in the formulation buffer. The pI and stability of durvalumab was determined using capillary isoelectric focusing and high-pressure size exclusion chromatography (HPSEC), respectively.

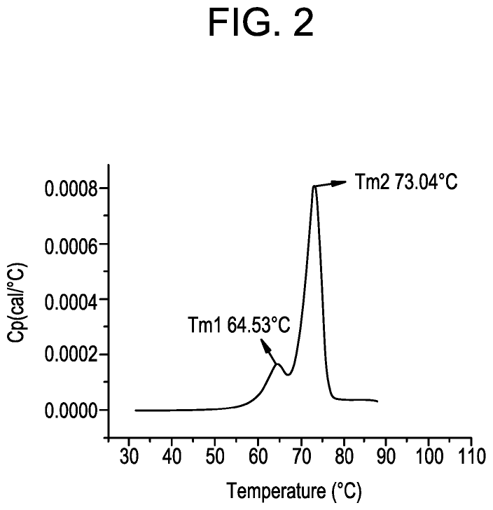

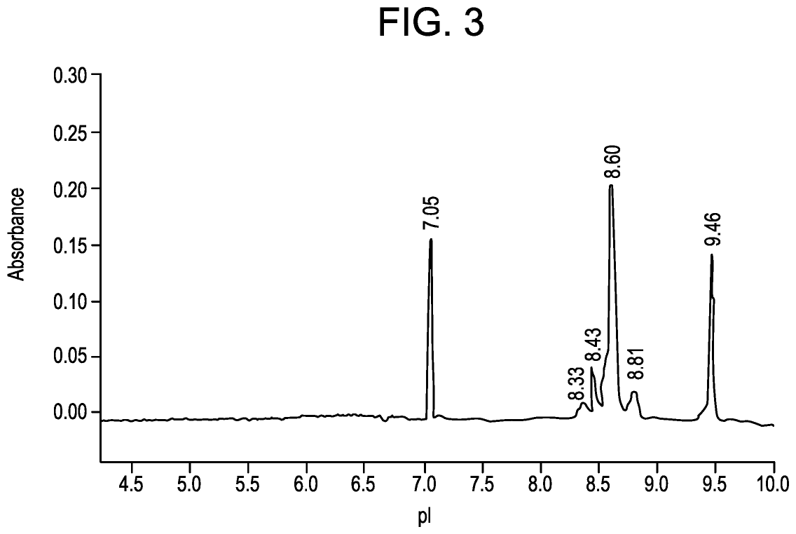

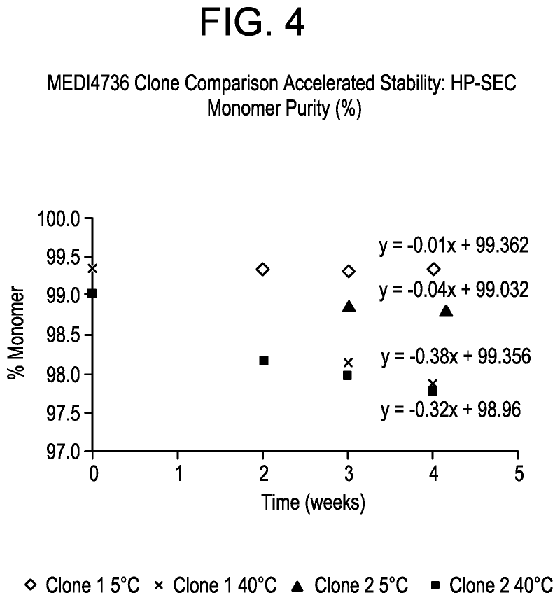

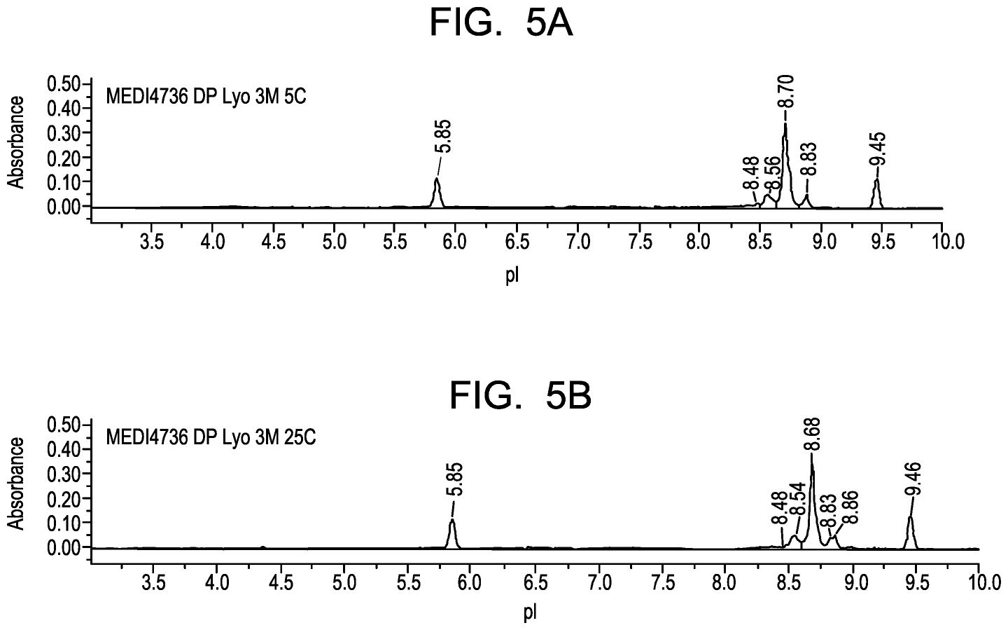

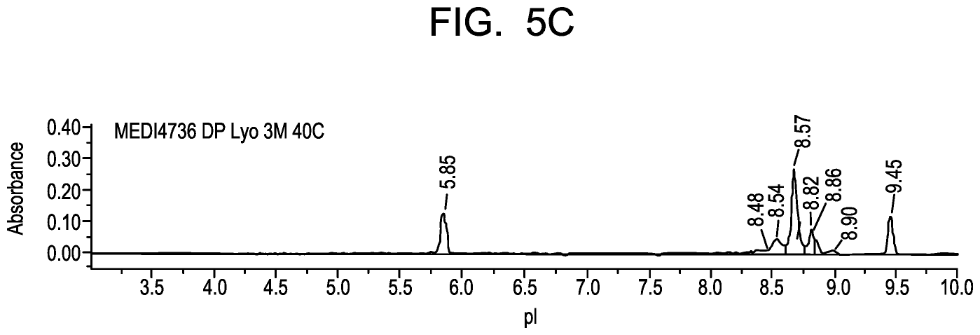

The DSC profile of durvalumab was shown as Tm₁ at 64.5° C. and Tm₂ at 73.04° C. (FIG. 2). Four peaks, ranging from 8.3-8.8 were detected in the capillary Isoelectric focusing (cIEF) profile of durvalumab. The main peak had an isoelectric point of 8.6 (FIG. 3). Liquid stability studies revealed that clone 1 and clone 2 were stable in default formulation following 1 month incubation at either 5° C. or 40° C. (FIG. 4). Developability study results are summarized in Table 1.

TABLE 1
Developability Study Results

| Description | Result |
|---|---|
| Formulation | 26 mM histidine/histidine-HCl, 275 mM |
| | trehalose dehydrate, 0.02% (w/v) P80, pH 6.0 |
| Clones | 130.174 (SP11-006), 272.253 (SP10-011), no |
| | significant difference between the two clones. |
| | Clone 130.174 selected for development |
| Capillary DSC | Tm₁: 64.5° C., Tm₂: 73.0° C. (FIG. 2) |
| cIEF | Main peak: 8.6, Range 8.3-8.8, Number of |
| | Peaks: 4 (FIG. 3) |
| Accelerated Stability | 40° C.: ~1 month |
| (1 Month, HP-SEC) | 2-8° C.: no change |
| | Primary Degradation route: aggregation |
| Visual Inspection | Practically free from visible particles |

### Example 3

### Lyophilized Formulation

Five formulations were screened to assess liquid drug substance and lyophilized drug product stability. The formulations that were screened are shown in Table 2.

An initial level of P80 (i.e., 0.02% w/v) was used. During subsequent formulation screening for the lyophilized formulation 0.04% (w/v) P80 (Formulation 4) was also included in case particles were observed upon reconstitution of the 0.02% (w/v) P80 containing formulation. Based on this study's data, 0.02% (w/v) P80 was found to be the optimal surfactant level for durvalumab.

TABLE 2
Formulations Screened

| Formulation No. | Formulation |
|---|---|
| 1 | 25 mM Histidine/histidine-HCl, 265 mM trehalose |
| | dihydrate, 0.02% (w/v) P80, pH 5.5 |
| 2 | 26 mM Histidine/histidine-HCl, 275 mM trehalose |
| | dihydrate, 0.02% (w/v) P80, pH 6.0 |
| 3 | 25 mM Histidine/histidine-HCl, 265 mM trehalose |
| | dihydrate, 0.02% (w/v) P80, pH 6.5 |
| 4 | 25 mM Histidine/histidine-HCl, 265 mM trehalose |
| | dihydrate, 0.04% (w/v) P80, pH 6.0 |
| 5 | 25 mM Histidine/histidine-HCl, 265 mM sucrose, |
| | 0.02% (w/v) P80, pH 6.0 |

Formulations were lyophilized for a formulation screening study. Formulations underwent freezing, annealing, primary drying, and secondary drying in a Virtis Genesis 25EL Freeze-Dryer. The lyophilization cycle parameters are supplied in Table 3.

TABLE 3
Lyophilization Cycle Parameters for
Lyophilized Formulation Screening

| | Shelf Temperature | Pressure | Time |
|---|---|---|---|
| Step | (° C.) | (mTorr) | (minutes) |
| Freeze | −40 | N/A | 120 |
| Anneal | −8 | N/A | 60 |
| Primary Dry | −25 | 100 | Pressure Rise |
| | | | Test Control |
| Secondary Dry | 20 | 200 | 720 |

### 1. Stability

After 1 month at 40° C., all the lyophilized formulations had monomer loss rates of <0.3% per month (Table 4A). At 5° C., the rate of monomer loss per month for all formulations was <0.1% per month. All formulations were equivalent in terms of aggregation after 4 weeks at 40° C. (Table 4B). Aggregation was not present in any of the formulations after 4 weeks at 40° C. (Table 4C).

A freeze-thaw study was performed on all five formulations. Formulations underwent 0, 1, and 3 freeze-thaw cycles consisting of freezing at −70° C. and thawing at ambient temperature. Samples were visually inspected and analyzed via HP-SEC at the conclusion of the cycles. HP-SEC results revealed no significant change in purity by HP-SEC in any sample. All samples were virtually free of visible particles after three cycles.

TABLE 4A
Percent Monomer Formation of durvalumab Formulations

| | | % Monomer/Weeks | | | Trend | |
|---|---|---|---|---|---|---|
| Sample | Temperature. | 0 | 2 | 4 | Weekly | Monthly |
| Formulation 1 | 40° C. | 99.41 | 99.12 | 99.16 | −0.06 | −0.25 |
| Formulation 2 | 40° C. | 99.38 | 99.17 | 99.11 | −0.07 | −0.27 |
| Formulation 3 | 40° C. | 99.44 | 99.24 | 99.18 | −0.07 | −0.26 |
| Formulation 4 | 40° C. | 99.32 | 99.17 | 99.13 | −0.05 | −0.20 |
| Formulation 5 | 40° C. | 99.40 | 99.31 | 99.30 | −0.03 | −0.10 |

TABLE 4B
Percent Aggregation of durvalumab Formulations

| | | % Aggregate/Weeks | | | Trend | |
|---|---|---|---|---|---|---|
| Sample | Temperature | 0 | 2 | 4 | Weekly | Monthly |
| Formulation 1 | 40° C. | 0.59 | 0.88 | 0.84 | 0.06 | 0.25 |
| Formulation 2 | 40° C. | 0.62 | 0.83 | 0.89 | 0.07 | 0.27 |
| Formulation 3 | 40° C. | 0.56 | 0.76 | 0.82 | 0.07 | 0.26 |
| Formulation 4 | 40° C. | 0.68 | 0.83 | 0.87 | 0.05 | 0.20 |
| Formulation 5 | 40° C. | 0.60 | 0.69 | 0.7 | 0.03 | 0.10 |

TABLE 4C
Percent Aggregation of durvalumab Formulations

| | | % Fragment/Weeks | | | Trend | |
|---|---|---|---|---|---|---|
| Sample | Temperature | 0 | 2 | 4 | Weekly | Monthly |
| Formulation 1 | 40° C. | 0.00 | 0.00 | 0.00 | 0.00 | 0.00 |
| Formulation 2 | 40° C. | 0.00 | 0.00 | 0.00 | 0.00 | 0.00 |
| Formulation 3 | 40° C. | 0.00 | 0.00 | 0.00 | 0.00 | 0.00 |
| Formulation 4 | 40° C. | 0.00 | 0.00 | 0.00 | 0.00 | 0.00 |
| Formulation 5 | 40° C. | 0.00 | 0.00 | 0.00 | 0.00 | 0.00 |

### 2. Post-Reconstitution Stability

All five lyophilized formulations were tested for stability after reconstitution for up to 24 hours of storage at 5° C. and 25° C. None of the five formulations demonstrated a change in number of visible particles or a significant decrease in monomer purity, as assessed by SEC-HPLC. Results at TO are shown in Table 5A, 24 hours at 5° C. in Table 5B, and 24 hours at 25° C. in Table 5C.

TABLE 5A
Post Reconstitution Stability (T0)
T0

| Formulation | 1 | 2 | 3 | 4 | 5 |
|---|---|---|---|---|---|
| Visual | Practically | Practically | Practically | Practically | Practically |
| Inspection | Free | Free | Free | Free | Free |
| SEC-HPLC | 99.4 | 99.4 | 99.4 | 99.4 | 99.4 |
| (% | | | | | |
| Monomer) | | | | | |

TABLE 5B
Post Reconstitution Stability (24 hr at 5° C.)
T = 24 hr 5° C.

| Formulation | 1 | 2 | 3 | 4 | 5 |
|---|---|---|---|---|---|
| Visual | Practically | Practically | Practically | Practically | Practically |
| Inspection | Free | Free | Free | Free | Free |
| SEC-HPLC | 99.4 | 99.4 | 99.4 | 99.4 | 99.4 |
| (% | | | | | |
| Monomer) | | | | | |

TABLE 5C
Post Reconstitution Stability (24 hr at 25° C.)
T = 24 hr 25° C.

| Formulation | 1 | 2 | 3 | 4 | 5 |
|---|---|---|---|---|---|
| Visual | Practically | Practically | Practically | Practically | Practically |
| Inspection | Free | Free | Free | Free | Free |
| SEC-HPLC | 99.4 | 99.4 | 99.3 | 99.4 | 99.4 |
| (% | | | | | |
| Monomer) | | | | | |

3. Freeze-Thaw Study for durvalumab in 26 mM Histidine/histidine-HCl, 275 mM trehalose dehydrate, 0.02% (w/v) polysorbate 80, pH 6.0 Formulation

Antibody structure degradation related to repeated freeze/thaw cycles was assessed to examine durvalumab stability. Consistent with this, the stability of durvalumab was tested using freeze-thaw studies.

Durvalumab unformulated drug substance (UDS) freeze-thaw studies were performed on the formulation (26 mM Histidine/histidine-HCl, 275 mM trehalose dehydrate, 0.02% (w/v) polysorbate 80, pH 6.0). durvalumab underwent 0, 1, and 3 uncontrolled freeze-thaw cycles in 8 mL Nalgene bottles filled with 5.6 mL or in 30 mL Celsius Pak bags filled with 20 mL. Nalgene bottles were frozen at −80° C. and Celsius Pak bags at −40° C. with thawing of both at ambient temperature. Samples were then analyzed for purity by High Pressure Size Exclusion Chromatography (HP-SEC) Analysis, protein concentration determined by A280, and visual appearance assessed after each freeze-thaw cycle.

Durvalumab was found to be stable after 3 freeze-thaw cycles as show in Table 6A (Nalgene Bottles) and 6B (Celsius Paks) below:

TABLE 6A
Durvalumab Freeze/Thaw Results in Nalgene Bottles

| Freeze/ | | | | |
|---|---|---|---|---|
| Thaw | HPSEC | Visual Inspection | | A280 |
| Cycle | (% Monomer) | Operator 1 | Operator 2 | (mg/mL) |
| 0 | 99.3% | Practically Free | Practically Free | 49.8 |
| 1 | 99.3% | Practically Free | Practically Free | 50.4 |
| 3 | 99.3% | Practically Free | Practically Free | 50.5 |

TABLE 6B
Durvalumab Freeze/Thaw Results in Celsius Paks

| Freeze/ | | | | |
|---|---|---|---|---|
| Thaw | HPSEC | Visual Inspection | | A280 |
| Cycle | (% Monomer) | Operator 1 | Operator 2 | (mg/mL) |
| 0 | 99.3% | Practically Free | Practically Free | 49.8 |
| 1 | 99.3% | Practically Free | Practically Free | 50.9 |
| 3 | 99.3% | Practically Free | Practically Free | 50.7 |

These results indicate durvalumab was stable following repeated freeze/thaw cycles in both Nalgene bottles and Celsius Paks.

### Example 4

### Pyroglutamic Acid Formation & Detection

Durvalumab has an N-terminal glutamic acid on the heavy and light chains. Over time, the glutamic acid cyclizes to pyroglutamic acid. This conversion was detected in the cIEF assay as a peak at pI 8.8 after three months of storage at 25° C. and 40° C. The peak occurred at 2-8° C. after 24 months as well (FIG. 4). The cyclisation occurred in both the lyophilized and liquid forms. A mutated durvalumab was created that showed that the cyclization had no effect on potency of the antibody.

### Example 5

### Final Lyophilized Formulation Parameters and Stability

The 26 mM histidine/histidine-HCl, 275 mM trehalose dehydrate, 0.02% (w/v) polysorbate 80, pH 6.0 lyophilized formulation contained 50 mg/mL durvalumab. Parameters of the 26 mM histidine/histidine-HCl, 275 mM trehalose dehydrate, 0.02% polysorbate 80, pH 6.0 formulation are shown in Table 7, and stability of the lyophilized drug product in the final formulation is provided in Table 8. The solubility profile of the lyophilized drug substance in final formulation in polypropylene tubes is presented in Table 9.

TABLE 7
Drug Product Parameters of 26 mM histidine/histidine-HCl, 275 mM
trehalose dehydrate, 0.02% polysorbate 80, pH 6.0 Formulation

| Parameter | Value |
|---|---|
| Container & Closure System | 10R borosilicate glass vial, |
| | bromobutyl lyo stopper, 20 mm |
| | Flip-Cap Aluminum overseal |
| Fill volume (mL) | 4.5 |
| Extractable Volume (mL) | ~4.0 |
| Drug substance stability for x | x = 3 |
| freeze/thaw cycles | |
| Concentration (mg/mL) | 50 |
| Density (g/cm³) | 1.05 |
| Osmolality (mOsm) | 360 |
| pH | 6.0 |
| Viscosity at 20° C. (mPa/sec) | IV Only |
| pI (Range) | 8.33-8.60 |
| pI (Number of Peaks) | 4 (+1 due to |
| | pyro-glutamic acid) |
| Tₘ₁ (° C.) | 64.7 |
| T_{g}' (° C.) | −27.1 |
| Reconstitution Volume (mL) | 4.0 |
| Reconstitution Time (min) | 5-10 mins |

TABLE 8
Stability Profile of Lyophilized Drug Product in Final Formulation

| | Storage Temperature & Length | | |
|---|---|---|---|
| | 5° C. | 25° C. | 40° C. |
| Assay | (24 months) | (6 months) | (6 months) |
| HPSEC (% monomer loss per | 0.01 | 0.04 | 0.17 |
| month) | | | |
| HPSEC (% aggregation per month) | 0.01 | 0.04 | 0.17 |
| HPSEC (% fragmentations per | 0.00 | 0.00 | 0.00 |
| month) | | | |
| Visual Inspection (Particles) | Std 1 | <Std 1 | <Std 1 |
| Visual Inspection (Clarity) | <Std 2 | <Std 2 | <Std 2 |
| Visual Inspection (Color) | Y6 | Y6 | Y6 |
| Reducing BioA (% heavy + | 99.7 | 98.7 | 100 |
| light chain + heavy chain | | | |
| leading peak) | | | |
| Non-reducing BioA (% intact IgG) | 100 | 99.5 | 99.5 |
| SVP (≥10 μm particles/mL) | 47 | 33 | 427 |
| SVP (≥25 μm particles/mL) | 0 | 7 | 20 |
| Karl Fischer (% water) | 0.3 | 0.4 | 0.4 |
| iCE (pI Range) | 8.37-8.84 | 8.30-8.83 | 8.32-9.01 |
| iCE (no. of peaks) | 5 | 5 | 5 |

TABLE 9
Solubility Profile of Lyophilized Drug Substance
in Final Formulation in Polypropylene Tubes

| | Storage Temperature & Length | | |
|---|---|---|---|
| | 5° C. | 25° C. | 40° C. |
| Assay | (24 months) | (6 months) | (6 months) |
| HPSEC (% monomer loss per | 0.02 | 0.22 | 2.08 |
| month) | | | |
| HPSEC (% aggregation per month) | 0.02 | 0.15 | 1.52 |
| HPSEC (% fragmentations per | 0.00 | 0.07 | 0.56 |
| month) | | | |
| Visual Inspection (Particles) | Std 1 | <Std 1 | <Std 1 |
| Visual Inspection (Clarity) | Std 1 | Std 1 | Std 1 |
| Visual Inspection (Color) | <Y5 | Y6 | Y4 |
| Reducing BioA (% heavy + | 99.8 | 96.2 | 100 |
| light chain + heavy chain | | | |
| leading peak) | | | |
| Non-reducing BioA (% intact IgG) | 99.6 | 99.3 | 99.5 |
| SVP (≥10 μm particles/mL) | 67 | 60 | 73 |
| SVP (≥25 μm particles/mL) | 7 | 40 | 0 |
| iCE (pI Range, main peak) | 8.35-8.83 | 8.23-8.80 | 8.08-8.69 |
| | (8.68) | (8.59) | (8.40) |
| iCE (no. of peaks) | 5 | 4 | 5 |

### Example 6

### Lyophilization Cycle Development

The lyophilization cycle development resulted in the parameters shown in Table 10.

TABLE 10
Overview of Lyophilization Cycle Development Results

| Description | Results | |
|---|---|---|
| Formulation | 26 mM histidine/histidine-HCl, | |
| | 275 mM trehalose dehydrate, | |
| | 0.02% (w/v) polysorbate 80, pH 6.0 | |
| Tg' by DSC (standard, not | −27.14° | C. |
| modulated) | | |
| Tc by freeze-dry microscope | Tc, onset: −33.0° C., | |
| | Tc, full: −27.3° C. | |
| Freeze-dryer used for cycle | Virtis Genie 25EL | |
| development | | |
| End-point of primary drying | Pressure Test Rise | |
| method | | |
| Container closure | 10R vials, 20 mM Stelmi stoppers | |
| Fill volume | 4.5 | mL |

| | |
|---|---|
| Drying time target | <7 days, conservative cycle |

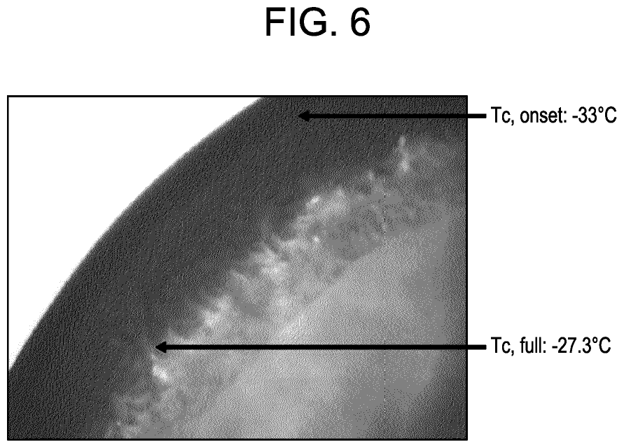

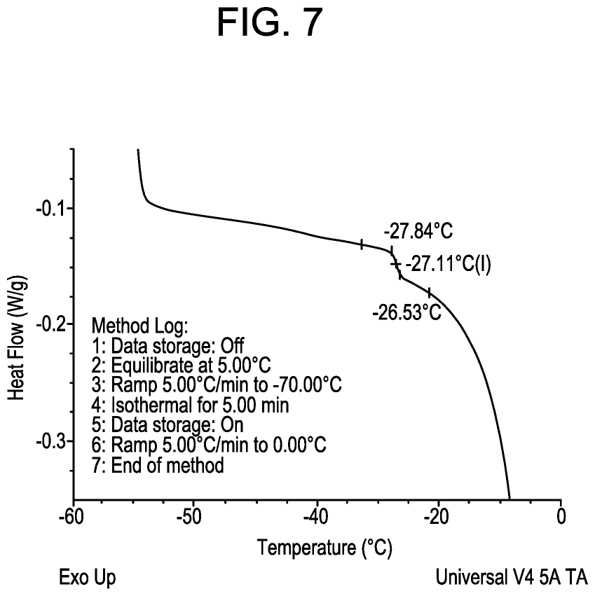

Freeze-dry microscopy was used to assess the lyophilization cycle temperature of collapse onset (−33° C.) and full collapse (−27.3° C.; FIG. 6). The glass transition temperature (Tg′) was determined to be −27.14° C. by Differential Scanning calorimetry (DSC; FIG. 7).

A design space model of the lyophilization process was produced using Design Expert 7 with a design-of-experiments (DoE) approach. The DoE approach was used to achieve a robust, conservative cycle of <7 days. The run conditions chosen, representing the midpoint (around the Tg′) and the four ‘corners’ of the experimental space, are set forth in Table 11.

TABLE 11
Experimental Design (DoE) for durvalumab
Lyo Cycle Development

| | | Primary | | Primary | |
|---|---|---|---|---|---|
| | | Drying | | Drying | Max Product |
| Standard | Run Order | Temp | Pressure | Time | Temperature |
| Order | (randomized) | (° C.) | (mTorr) | (h) | (° C.) |
| 5 | 1 | −28 | 100 | 90.3 | −29.5 |
| 1 | 2 | −33 | 65 | 138.8 | −33.2 |
| 3 | 3 | −33 | 135 | 227.4 | −33.5 |
| 2 | 4 | −23 | 65 | 67.8 | −28.3 |
| 4 | 5 | −23 | 135 | 63.9 | −26.5 |

All experimental cycles had the same ramp rates, freeze temperatures, and secondary drying conditions as described in the final cycle. The final durvalumab lyophilization cycle conditions are shown in Table 12.

TABLE 12
Durvalumab Cycle 1 Final Lyophilization Cycle Parameters

| | Heat/ | Temp | Time | Pressure | Time | Rate |
|---|---|---|---|---|---|---|
| Step | Ramp | (° C.) | (mins) | (mBar) | (h) | (° C./min) |
| Freeze | H | 20 | 60 | — | 1 | |
| | R | −40 | 90 | — | 1.5 | 0.67 |
| | H | −40 | 120 | — | 2 | |
| | R | −10 | 60 | | 1 | 0.5 |
| | H | −10 | 120 | | 2 | |
| | R | −40 | 60 | | 1 | 0.5 |
| | H | −40 | 120 | | 2 | |
| Vacuum on | H | −40 | 30 | 105 | 0.5 | |
| 1° drying | R | −25 | 31 | 105 | 0.52 | 0.48 |
| | H | −25 | 6900 | 105 | 115 | |
| | R | 20 | 225 | 105 | 3.75 | 0.20 |
| 2° drying | H | 20 | 720 | 105 | 12 | |
| Stoppering | | 5 | | 850 | | |
| Unloading | | 20 | | Ambient | | |

| | |
|---|---|
| Total | 8536 mins, 142.3 hours, 5.9 days |

The main criteria in developing a lyophilization cycle were: (1) Maintain product temperature above the collapse temperature (roughly equivalent to Tg′); (2) ensure primary drying time was no more than 115 hours in order to keep cycle time less than six days; (3) provide sufficient robustness for scale-up (2-3° C. shelf temperature and 40m Torr chamber pressure).

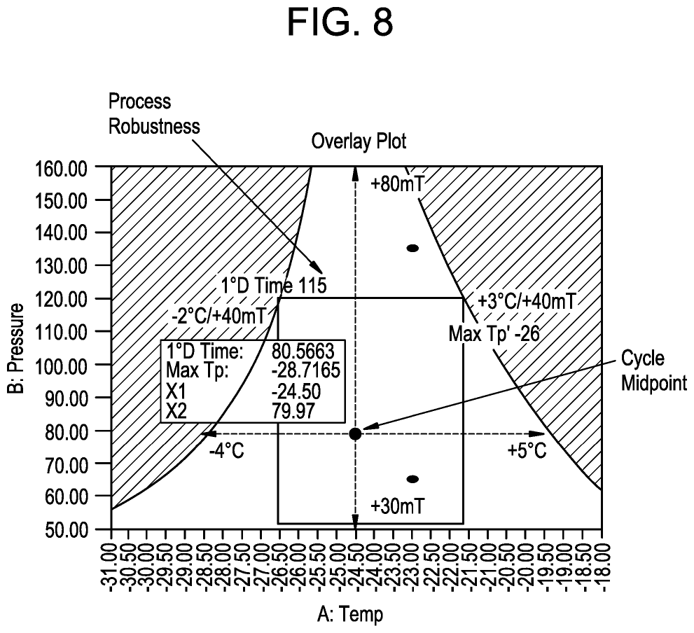

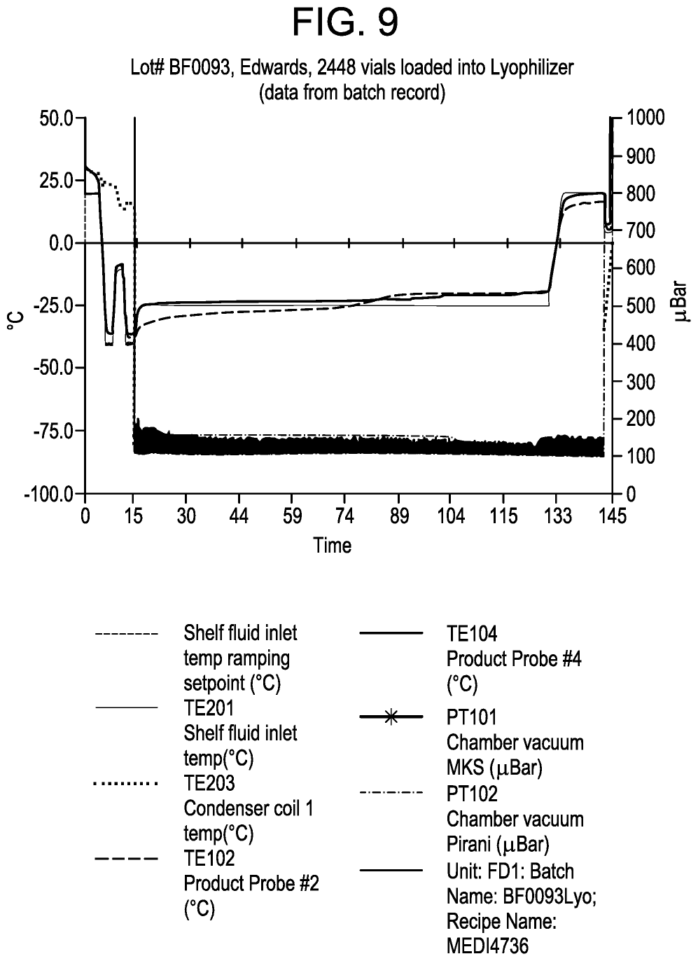

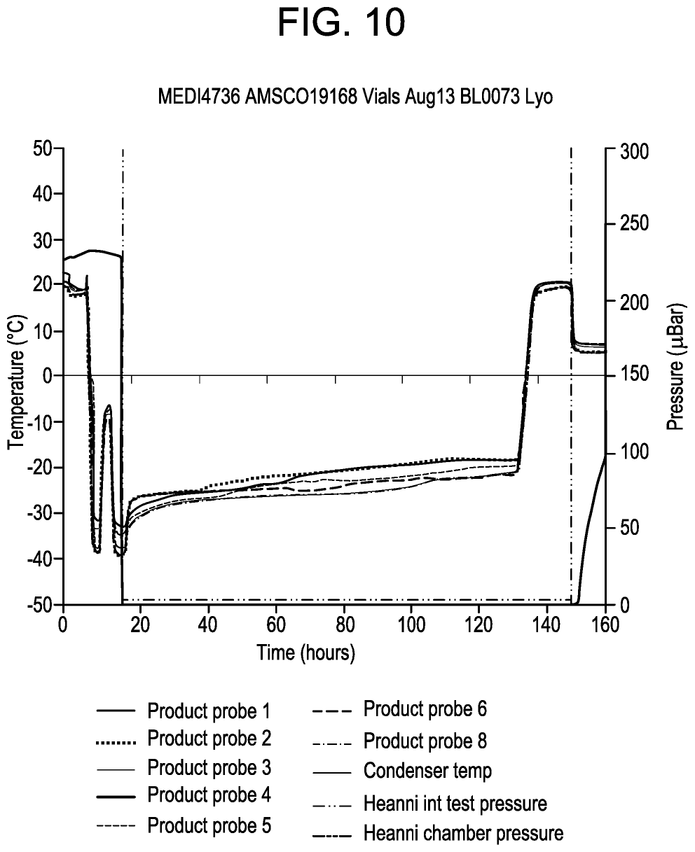

Experimental runs in Table 11 resulted in the design space of the lyophilization process. The design space generated from the five experimental runs is depicted in FIG. 8. Briefly, cycle midpoint and process robustness around chamber and shelf temperature were determined. Data from NMF Edwards freeze-dryer showed convergence of the pirani gauge (delta 10 ubar) at approximately 103 hours into the 115-hour primary drying step. This is equivalent to an approximate 10% safety margin (FIG. 9). This is consistent with a previous, large-scale run, in which an Amsco freeze-dryer showed convergence of the product thermocouples within the allotted primary time (FIG. 10).

### Example 7

### Liquid Formulation Development

### 1. Shaking Study for Optimization of Surfactant Levels

Liquid formulation suitability of polysorbate 80 was assessed via a shaking study in vials. Samples containing 0, 0.005%, 0.01%, 0.02%, 0.03%, 0.04%, and 0.05% (w/v) polysorbate 80 were placed in tubes with a worst-case air to liquid volume ration of 6.5 mL. Tubes were vigorously agitated at 600 rpm for four hours at ambient temperature. Samples were then inspected by visual analysis, A280, BioA, HP-SEC, and micro-flow imaging (MFI). Control unshaken tubes placed at 2-8° C. were also analyzed.

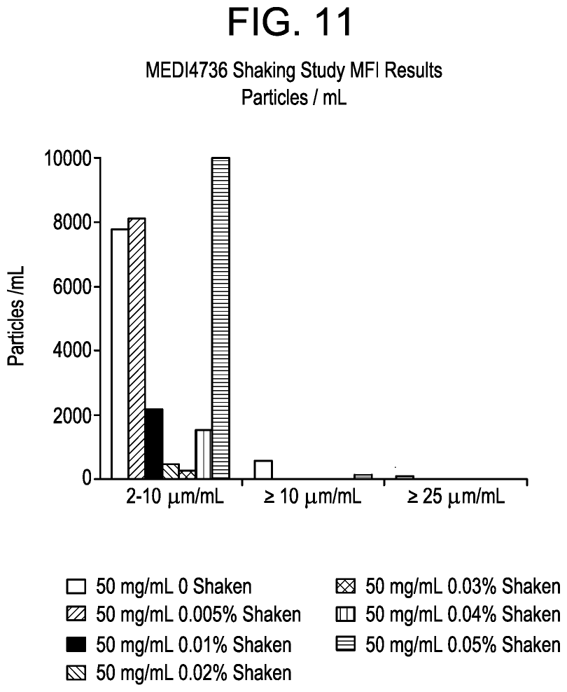

No observable changes were noted in percent purity as analyzed by BioA and HP-SEC. No changes in A280 protein concentration resulted from shaking. MFI analysis revealed that all samples had low levels of >10 μm and >25 μm sub-visible particles (FIG. 11). Sub-visible particle levels were overall low in all tubes, with 0.02% polysorbate 80 chosen as the optimal surfactant level for the liquid formulation.

### 2. Controlled Freeze-Thaw Study

The 26 mM histidine/histidine-HCl, 275 mM trehalose dehydrate, 0.02% polysorbate 80, pH 6.0 formulation underwent five controlled freeze-thaw cycles, thawing at 5° C. and freezing at −40° C. At the conclusion of the fifth freeze/thaw cycle purity, visual appearance, sub-visible particles, and protein concentration were determined. No significant change in product quality following the fifth freeze-thaw cycle was observed (Table 13).

TABLE 13
Freeze/Thaw Cycle Results (26 mM histidine/histidine-HCl, 275 mM
trehalose dehydrate, 0.02% polysorbate 80, pH 6.0 Formulation)

| | | |
|---|---|---|
| | | After 5x freeze- |
| Assay | T = 0 | thaw cycles |

| | |
|---|---|
| Formulation | 26 mM histidine/histidine-HCl, |
| | 275 mM Trehalose dehydrate, |
| | 0.03% (w/v) polysorbate 80, pH 6.0 |

| | | |
|---|---|---|
| Purity (%) by HP-SEC | 99.7 | 99.7 |
| Visual Appearance | <Std1 | Std1 |
| Sub-visible particles | 2-10 μm: 8960 | 2-10 μm: 21840 |
| by HIAC (Particles per | ≥10 μm: 630 | ≥10 μm: 980 |
| 10.5 mL container) | ≥25 μm: 0 | ≥25 μm: 70 |
| Protein Concentration | 51.8 | 52.8 |
| by A280 (mg/mL) | | |

### 3. Formulation Stability Summary

The stability profile of the lyophilized drug product in the final formulation is provided in Table 14. The results demonstrate that lyophilized durvalumab in the final formulation remains stable after prolonged storage.

TABLE 14
Stability Profile of Lyophilized Drug
Product in the Final Formulation

| | Storage Temperature & Length | | |
|---|---|---|---|
| | 5° C. | 25° C. | 40° C. |
| Assay | (6 months) | (6 months) | (3 months) |
| HPSEC (% monomer loss per month) | 0.05 | 0.4 | 2.3 |
| HPSEC (% aggregation per month) | 0.05 | 0.3 | 1.6 |
| HPSEC (% fragmentations per | 0.00 | 0.1 | 0.7 |
| month) | | | |
| Visual Inspection (Particles) | Std 1 | Std1 | <Std2 |
| Visual Inspection (Clarity) | <Std 2 | <Std2 | <Std2 |
| Visual Inspection (Color) | Y6 | Y5 | Y3 |
| Reducing BioA (% heavy + | 99.6 | 98.4 | 98.7 |
| light chain + heavy chain | | | |
| leading peak) | | | |
| Non-reducing BioA (% intact IgG) | 99.6 | 97.6 | 93.5 |
| SVP (≥10 μm particles/mL) | 73 | 33 | 13 |
| SVP (≥25 μm particles/mL) | 0 | 7 | 0 |
| iCE (pI Range) | 8.3-8.84 | 8.22-8.81 | 8.17-8.84 |
| iCE (no. of peaks) | 4 | 5 (4 at | 6 |
| | | T = 3 m) | |

### Example 8

### N-Linked Oligosaccharide Identification

The Fc region of durvalumab contains an N-linked oligosaccharide chain attached to a single site on the heavy chain at Asn-301. The structure characterization of the oligosaccharides on durvalumab is critical to the understanding of the structural micro heterogeneity of the product. It is also important for quality control when the process changes.

The oligosaccharides cleaved from durvalumab that were present in the 2-AB labelled N-linked oligosaccharides profile by ultra-performance liquid chromatography (UPLC) were characterized, including the low abundance glycoforms. Digestion of 2-AB labelled oligosaccharides with exo-glycosidases as described in Table 15 was performed to verify non-reducing terminal monosaccharide residues. Oligosaccharide profiling was completed as shown in Table 16. LC/MS analysis was used to verify the molecular weight.

TABLE 15
Sample Preparation for durvalumab Oligosaccharide Digestion

| | Enzymes | | | | | |
|---|---|---|---|---|---|---|
| | | | | | β- | β--N-Acetyl |
| Reagents | Control | Fucosidase | Sialidase A | Mannosidase | Galactosidase | hexosaminidase |
| 2-AB labeled durvalumab | 16 | 16 | 16 | 16 | 16 | 16 |
| glycans (μL) | | | | | | |
| Reaction buffer (μL) | 4 | 4 | 4 | 4 | 4 | 4 |
| Exo-glycosidase (μL) | 0 | 4 | 2 | 2 | 2 | 2 |
| Water (μL) | 2 | 0 | 0 | 0 | 0 | 0 |
| Total Volume (μL) | 22 | 24 | 22 | 22 | 22 | 22 |

TABLE 16
Gradient of UPLC Oligosaccharide Profiling

| Time | % A | % B |
|---|---|---|
| Initial | 27 | 73 |
| 23 | 41 | 59 |
| 23.1 | 100 | 0 |
| 27.5 | 100 | 0 |
| 27.6 | 27 | 73 |
| 30 | 27 | 73 |

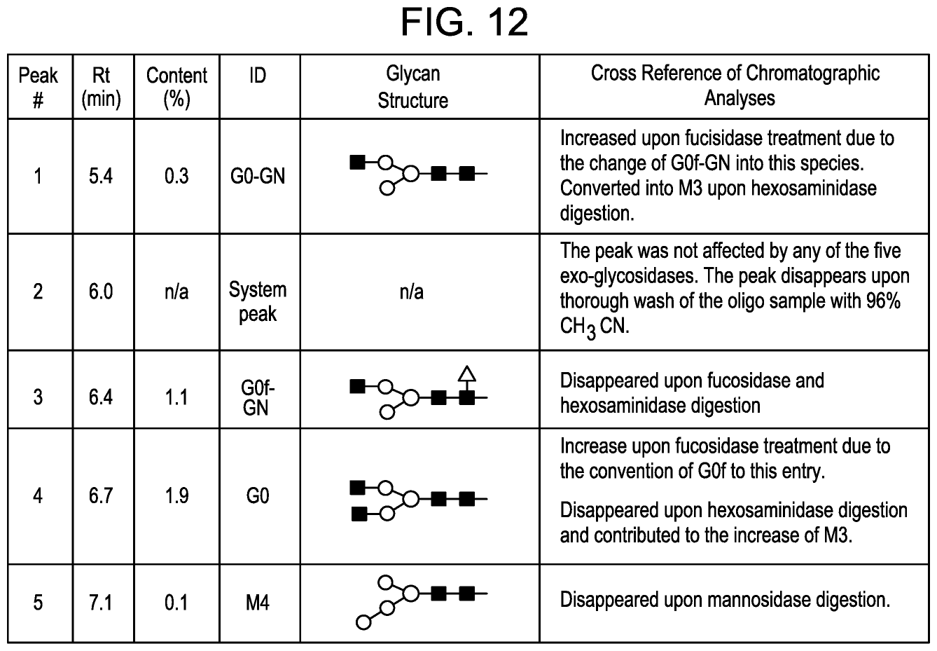

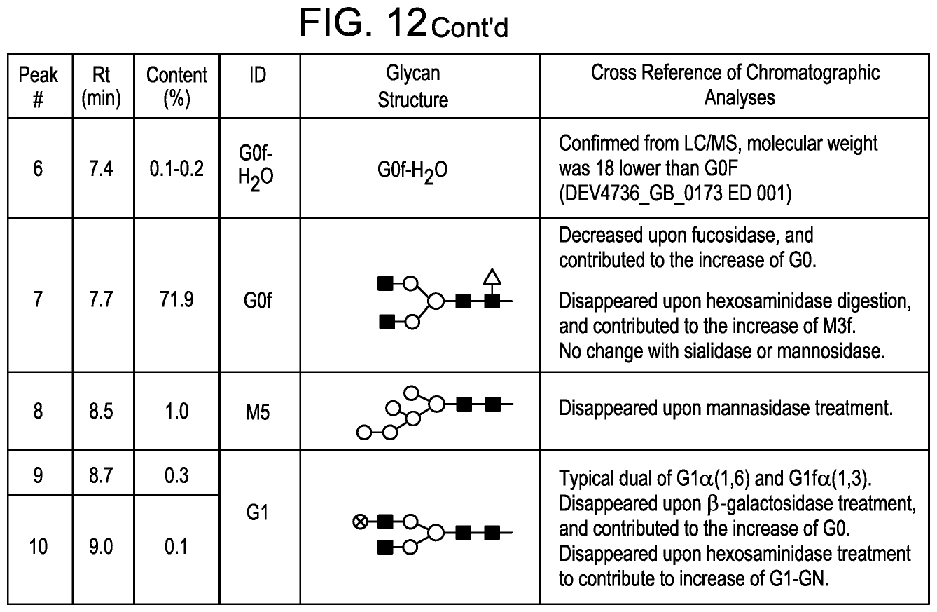

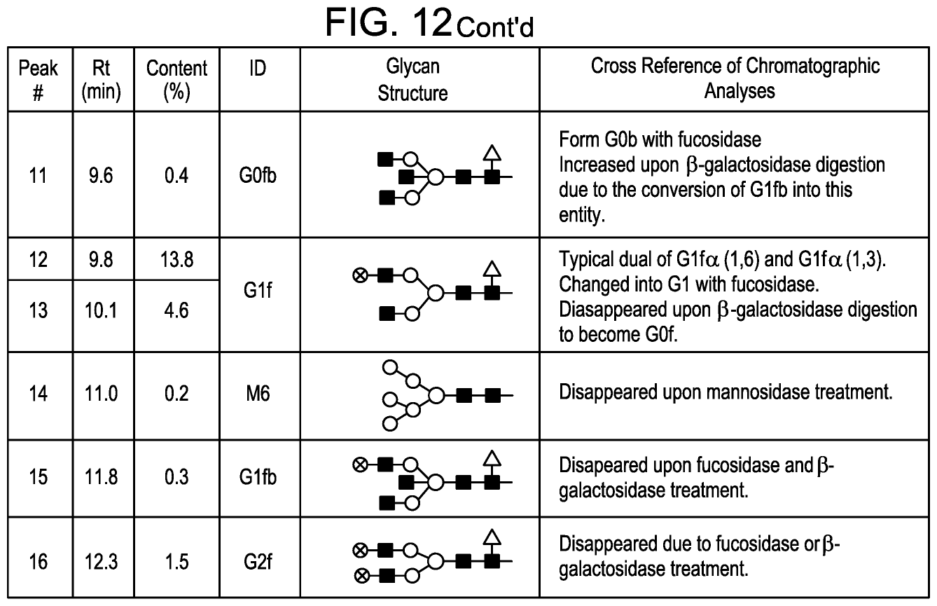

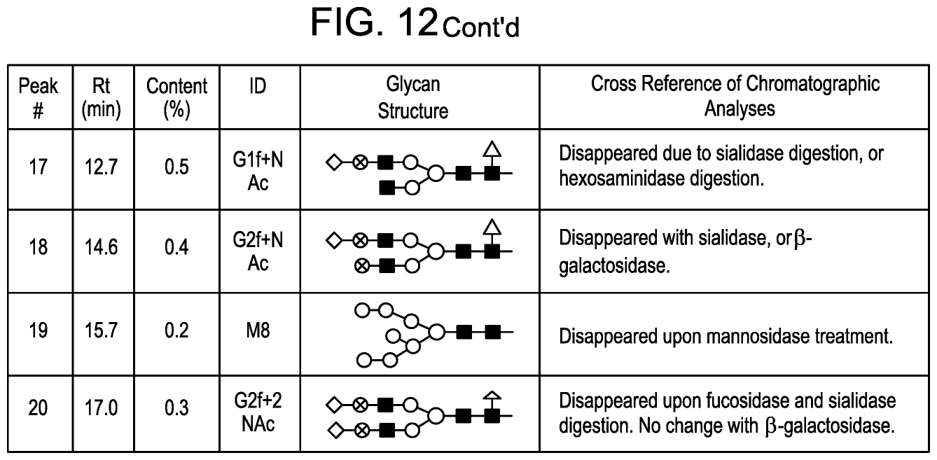

The N-linked oligosaccharides in durvalumab that were detected as significant peaks in UPLC were identified and the results shown in FIG. 12. The predominant glycoforms of durvalumab are fucosylated biatennary complex-type oligosaccharides with either no terminal galactose residues (G0f), or mono-galactosylated (G1f), and di-galactosylated (G2f) forms. The minor complex glycoforms were afucosylated G0 and G1, truncated G0f and G0 forms without N-acetyl-glucosamine (GlcNAc), sialylated G1f and G2f forms (G1f+NAc, G2f+NAc, or G2f+2NAc), and bisecting structures G0fb and G1fb. Depending on the maturity of the Chinese Hamster Ovary (CHO) cell when IgG was harvested, high mannose glyocforms (Man4, Man5, Man6, Man7, and Man8) were also present.

It should be understood that the foregoing disclosure emphasizes certain specific embodiments of the invention and that all modifications or alternatives equivalent thereto are within the spirit and scope of the invention as set forth in the appended claims.

TABLE OF SEQUENCES

| | |
|---|---|
| SEQ ID NO: 1 | EIVLTQSPGTLSLSPGERATLSCRASQRVSSSYL |
| Human Anti-PD-L1 mAb (durvalumab)- | AWYQQKPGQAPRLLIYDASSRATGIPDRFSGSGS |
| VL polypeptide sequence | GTDFTLTISRLEPEDFACYYCQQYGSLPWTFGQG |
| | TKVEIK |
| SEQ ID NO: 2 | EVLQVESGGGLVQPGGSLRLSCAASGFTFSRYWM |
| Human Anti-PD-L1 mAb (durvalumab)- | SWVRQAPGKGLEWVANIKQDGSEKYYVDSVKGRF |
| VH polypeptide sequence | TISRDNAKNSLYLQMNSLRAEDTAVYYCAREGGW |
| | FGELAFDYWGQGTLVTVSS |
| SEQ ID NO: 3 | GFTFSRYWMS |
| Human Anti-PD-L1 mAb (durvalumab)- | |
| VH CDR1 polypeptide sequence | |
| SEQ ID NO: 4 | NIKQDGSEKYYVDSVKG |
| Human Anti-PD-L1 mAb (durvalumab)- | |
| VH CDR2 polypeptide sequence | |
| SEQ ID NO: 5 | EGGWFGELAFDY |
| Human Anti-PD-L1 mAb (durvalumab)- | |
| VH CDR3 polypeptide sequence | |
| SEQ ID NO: 6 | |
| Human Anti-PD-L1 mAb (durvalumab)- | RASQRVSSSYLA |
| VL CDR1 polypeptide sequence | |
| SEQ ID NO: 7 | DASSRAT |
| Human Anti-PD-L1 mAb (durvalumab)- | |
| VL CDR2 polypeptide sequence | |
| SEQ ID NO: 8 | QQYGSLPWT |
| Human Anti-PD-L1 mAb (durvalumab)- | |
| VL CDR3 polypeptide sequence | |

1. An antibody formulation comprising:
(a) 40 mg/mL to 50 mg/mL of an anti-PD-L1 antibody;
(b) 15 mM to 35 mM buffer;
(c) 255 mM to 275 mM disaccharide; and
(d) 0.01% (w/v) to 0.05% (w/v) surfactant; and
wherein the pH of the formulation is pH 5.5 to pH 7.2.

2. The antibody formulation of claim 1, wherein the anti-PD-L1 antibody comprises:
(a) a light chain variable domain comprising the amino acid sequence of SEQ ID NO: 1, and a heavy chain variable domain comprising the amino acid sequence of SEQ ID NO: 2; or
(b) a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

3. The antibody formulation of claim 1, wherein the buffer is histidine/histidine-HCl buffer.

4. The antibody formulation of claim 1, wherein the disaccharide is trehalose dihydrate.

5. The antibody formulation of claim 1, wherein the disaccharide is sucrose.

6. The antibody formulation of claim 1, wherein the surfactant is polysorbate 80.

7. The antibody formulation of claim 1, wherein the formulation is a liquid formulation, a frozen formulation, a lyophilized formulation, or a reconstituted formulation.

8. The antibody formulation of claim 1, wherein less than about 1% of the anti-PD-L1 antibody forms an aggregate upon storage at 40° Celsius for about 1 month as measured by high-pressure size exclusion chromatography (HP-SPEC).

9. The antibody formulation of claim 1, wherein at least 97% of the anti-PD-L1 antibody is present as a monomer following storage at about 40° Celsius for about 1 month as measured by high-pressure size exclusion chromatography (HP-SEC).

10. The antibody formulation of claim 1, wherein at least 99% of the anti-PD-L1 antibody is present as a monomer following storage at about 40° Celsius for about 1 month as measured by high-pressure size exclusion chromatography (HP-SEC).

11. The antibody formulation of claim 1, wherein at least 98% of the anti-PD-L1 antibody is present as a monomer following storage at about 5° Celsius for about 1 month as measured by high-pressure size exclusion chromatography (HP-SEC).

12. The antibody formulation of claim 1, wherein the antibody formulation maintains stability following at least three freeze/thaw cycles.

13. An antibody formulation comprising:
(a) 50 mg/mL of a human anti-PD-L1 antibody;
(b) 26 mM histidine/histidine-HCl buffer;
(c) 275 mM trehalose dihydrate; and
(d) 0.02% (w/v) polysorbate 80; and
wherein the pH of the formulation is pH 6.0.

14-17. (canceled)

18. The antibody formulation of claim 13, wherein the human anti-PD-L1 antibody comprises a light chain having the amino acid sequence of SEQ ID NO: 1 and a heavy chain having the amino acid sequence of SEQ ID NO: 2.

19. The antibody formulation of claim 13, wherein the human anti-PD-L1 antibody comprises:
a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

20. The antibody formulation of claim 13, wherein the formulation is a liquid formulation, a frozen formulation, a lyophilized formulation, or a reconstituted formulation.

21. The antibody formulation of claim 13, wherein less than about 1% of the human anti-PD-L1 antibody forms an aggregate upon storage at 40° Celsius for about 1 month as determined by high-pressure size exclusion chromatography (HP-SPEC).

22. The antibody formulation of claim 13, wherein at least 97% of the human anti-PD-L1 antibody is present as a monomer following storage at about 40° Celsius for about 1 month as measured by high-pressure size exclusion chromatography (HP-SEC).

23. The antibody formulation of claim 13, wherein at least 99% of the human anti-PD-L1 antibody is present as a monomer following storage at about 40° Celsius for about 1 month as measured by high-pressure size exclusion chromatography (HP-SEC).

24. The antibody formulation of any one of claim 13, wherein at least 98% of the human anti-PD-L1 antibody is present as a monomer following storage at about 5° Celsius for about 1 month as measured by high-pressure size exclusion chromatography (HPSEC).

25. The antibody formulation of claim 13, wherein the antibody formulation maintains stability following at least three freeze/thaw cycles.

26-28. (canceled)

29. A composition comprising:
(a) an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2;
(b) a main form of the antibody comprising greater than, or equal to, 45% of the protein in the composition as measured using cIEF of the composition;
(c) acidic forms of the antibody comprising 45% to 50% of the protein in the composition as measured using cIEF of the composition; and
(d) a basic form of the antibody comprising 18% to 23% of the protein in the composition as measured using cIEF of the composition.

30. A composition comprising:
(a) an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2; and
(b) the glycan structures of the anti-PD-L1 antibody comprise G0f, G1f, G2f, and G0 glycoforms.

31. The composition of claim 30, wherein the glycan structures of the anti-PD-L1 antibody have a content greater than 90% for the G0f, G1f, G2f, and G0 glycoforms.

32. The composition of claim 30, wherein the anti-PD-L1 antibody comprises 71.9% G0f content, 18.4% G1f content, 1.5% G2f content, and 1.9% G0 content.

33. A composition comprising:
(a) an anti-PD-L1 antibody comprising a light chain having an amino acid sequence that is at least 90% identical to the amino acid sequence of SEQ ID NO: 1 and a heavy chain having an amino acid sequence that is at least 90% identical to the amino acid sequence of SEQ ID NO: 2;
(b) 1.5%-2.5% of the anti-PD-L1 antibody forms an aggregate as determined by high-pressure size exclusion chromatography (HP-SPEC); and
(c) 97%-98% of the anti-PD-L1 antibody is present as a monomer as measured by HP-SEC.

34. The composition of claim 33, wherein the PD-L1 antibody comprises:
a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

35. (canceled)
